(19) United States
(12) Patent Application Publication
(10) Pub. No.: US 2010/0304361 A1
(43) Pub. Date: Dec. 2, 2010

(54) METHOD AND DEVICE FOR MONITORING A THERAPEUTIC TREATMENT REGIME

(75) Inventors: Andrei Semikhodskii, London (GB); Simon Green, London (GB); Dale Smith, Kent (GB)

Correspondence Address: DINSMORE & SHOHL LLP, FIFTH THIRD CENTER, ONE SOUTH MAIN STREET, SUITE 1300, DAYTON, OH, 45402-2023, US

(73) Assignee: DAS DEVELOPMENT PARTNERS, London (GB)

(21) Appl. No.: 12/670,742

(22) PCT Filed: Jul. 28, 2008
(86) PCT No.: PCT/GB08/02581

§ 371 Date: Aug. 16, 2010

(30) Foreign Application Priority Data
Jul. 26, 2007 (GB) 0714598.0

(51) Int. Cl. C12Q 1/70; C12Q 1/68; C12M 1/34; G01N 33/571; G01N 33/569; G01N 33/50; G01N 33/48

(52) U.S. Cl. 435/5; 435/6; 435/287.1; 435/287.2; 435/7.36; 435/7.32; 436/94; 422/68.1

(57) ABSTRACT

A method for monitoring a therapeutic treatment regime in an individual having a disease, comprises: providing at a first location a solid substrate capable of immobilising a biomarker characteristic of the disease and a therapeutic compound in the biological sample, contacting the biological sample with the solid substrate to immobilise the biomarker and the therapeutic compound; transferring the solid substrate with the immobilised biomarker and therapeutic compound to a second location; performing an extraction step on the solid substrate to extract the biomarker and the therapeutic compound; performing a first detection assay to detect and/or quantify the biomarker, performing a second detection assay to quantify the therapeutic compound; and correlating the detection and/or quantity of the biomarker with the disease state of the individual, and comparing the quantity of the therapeutic compound with a target level for treatment of the disease, thereby to assess the efficacy of the treatment regime.

### FIELD OF THE INVENTION

The invention relates to kits and methods for monitoring the efficacy of medical regimes for the treatment of chronic infections. In particular, the invention relates to the use of a combination of pathogen and therapeutic drug detection in biological samples to monitor disease progression.

### BACKGROUND OF THE INVENTION

Since the identification in the nineteenth century of microorganisms as one of the major sources of morbidity and mortality, efforts have continued to monitor and control the spread of infectious disease. In the twentieth century, the advent of antibiotics, mass vaccination and antiviral treatments has offered an unprecedented level of control over the spread of such diseases, in the developed world at least. However, the widespread use of drugs for the therapeutic treatment and control of pathogens also brings its own problems; and in this respect, International health organisations such as the UN and the WHO consistently express concern over the unrestrained use of antibiotic compounds, leading to increasing levels of antibiotic resistance amongst many microbial species.

By way of example, there is a seemingly unrelenting succession of new or persistent microbial killers which challenge the medical profession, such as the multiple drug resistant bacteria, methicillin-resistant Staphylococcus aureus (MRSA) and Clostridium difficile (C. diff); Mycobacterium tuberculosis (TB); and highly virulent viral strains including the SARS coronavirus, avian flu and HIV; which are increasingly problematic to treat and/or cure using conventional techniques.

In addition, sexually transmitted infections (STIs) represent one of the greatest infectious disease problems in the world today. In some regions, particularly Africa and the former Soviet Union, STIs are at epidemic proportions. In fact, according to a recent study (Adler, M., 2005, Why sexually transmitted infections are important. In: ABC of Sexually Transmitted Infections. 5th ed. BMJ Books), the number of reported infected people in Western Europe was 17 million, in the USA it was 15 million, in Africa 70 million, and globally around 400 million. Hence, combating sexually transmitted infections, such as HIV/AIDS, remains uppermost on the world's governmental health agendas.

Despite the many significant medical breakthroughs that have been made in drug-based treatment regimes against pathogenic diseases, the continued spread of these diseases is evidence of the need for closer monitoring of disease states in infected individuals and in populations: for example, to reduce the risk of incubating new or known strains of drug-resistant pathogens.

There are many disease states of the human or animal body that can be monitored by measuring a marker of that disease, such as viral load in a patient infected with a viral pathogen. For example, in a patient infected with HIV, the concentration of HIV RNA (genome) detected in the patient's blood or plasma can be used to determine the progression of the disease. Hence, it is an aim of existing HIV therapies to maintain levels of plasma HIV at below 50 copies/ml: at which levels it is generally considered that the disease is under control. With this in mind, in known HIV-treatment regimes regular measurements of a patient's plasma HIV RNA levels are taken (e.g. once every 4-8 weeks), to monitor treatment efficacy. As noted, in HIV patients the regular measurement of plasma HIV RNA levels may be used to monitor drug efficacy and disease progress. Where HIV RNA levels are found to have increased relative to an earlier time point, this could be a sign that the virus is developing drug-resistance. Alternatively, it may indicate a lack of patient compliance with the treatment regime. Similar considerations, particularly with respect to disease progression in general, may apply to many other medical conditions, such as TB treatment.

The regular monitoring of patients in HIV-treatment programs is time-consuming and expensive, involving regular patient visits to hospitals or other medical centres for check-ups, collection of biological samples (e.g. blood/plasma), and HIV testing. Thus, the program may occupy several medical practitioners and laboratory technicians; and in financial terms, could cost up to $1000 per appointment.

Even with the time and money spent on patient monitoring programs, such as those for HIV, these programs can be inefficient, because patients may have to wait for long periods between consecutive check-ups; and ineffective where the root cause for any changes in the apparent disease state of an individual can not be determined.

Therapeutic drug monitoring (TDM: the measurement of the amount of a drug in a body fluid of a patient for the assessment of treatment efficacy), has been a controversial system; but with the move towards more individualised treatment regimes (e.g. “personal medicine”), it is now increasing in popularity. In TDM a quantity of a body fluid, e.g. blood, is extracted from a patient taking a particular medication, and the concentration of the relevant drug is measured. Typically, the measured concentration of the drug is then compared to a “target” (or recommended) concentration, or concentration range, of that drug for the treatment of the relevant disease state. A measured concentration of drug below the target level may be an indication that the medication is not providing its optimal therapeutic effect, while a level that is too high may warn of potential toxicological effects. It is also increasingly considered that TDM is important where a drug is known to have a narrow effective range.

However, TDM has a number of recognised limitations: for example, the detectable level of a drug can depend significantly on how long after the last drug treatment the biological sample is collected for testing. Furthermore, because individuals absorb, process and eliminate drugs at differing rates, blood levels may vary considerably amongst patients who have taken the same dose of medication. It is also important that TDM is correctly applied to the particular disease state under consideration. For example, when using an antibiotic to treat certain bacterial infections, it may be the peak plasma level of the drug that determines its therapeutic efficacy; whereas when treating HIV infection, for example, drug efficacy is often determined on the basis of the trough level of the medicament (i.e. the steady-state level of the drug).

Hence, it would be desirable to provide a means for monitoring the therapeutic efficacy of treatment regimes against pathogenic organisms that is reliable and/or relatively inexpensively. It would also be desirable to provide a means for the widespread monitoring of large numbers of out-patients, without causing a substantial drain on the resources of medical practitioners, staff and medical centres. It would further be desirable to provide a means for the reliable and safe testing of out-patients in their homes or otherwise in the field.

The present invention seeks to overcome or at least alleviate problems associated with the prior art.

### SUMMARY OF THE INVENTION

In broad terms, the present invention provides methods and kits suitable for high-throughput testing of biological samples from patients undergoing a treatment regime for a disease. The methods and kits provide for the combined measurement of a biomarker representative of a disease state and a therapeutic compound in a biological sample obtained from an individual, in order to monitor therapeutic efficacy of the therapeutic compound and progression of the disease state.

Accordingly, in a first aspect, the invention provides a method for monitoring a therapeutic treatment regime in an individual having a disease, comprising detecting and/or measuring the concentration of a biomarker characteristic of the disease and a therapeutic compound in a biological sample obtained from the individual, the method comprising the steps of: (a) providing at a first location at least one solid substrate, the at least one solid substrate capable of immobilising at least the biomarker and the therapeutic compound in the biological sample, and contacting the biological sample with the at least one solid substrate to immobilise the biomarker and the therapeutic compound on and/or within the solid substrate at the first location; (b) transferring the solid substrate with the immobilised biomarker and therapeutic compound to a second location; (c) performing an extraction step on the solid substrate to extract at least the biomarker and the therapeutic compound; (d) performing a first detection assay to detect and/or quantify the biomarker, and performing a second detection assay to quantify the therapeutic compound; and (e) correlating the detection and/or quantity of the biomarker with the disease state of the individual, and comparing the quantity of the therapeutic compound with a target level of the therapeutic compound for treatment of the disease, thereby to assess the efficacy of the treatment regime.

Thus, the method of the invention provides an advantageous system for assessing the efficacy of a treatment regime in an individual suffering from a disease by monitoring disease progression and correlate this with the bioavailable levels of the relevant therapeutic compound(s). By combining the measurement of TDM with the level of a particular biomarker associated with the disease state of interest (e.g. HIV RNA concentration), it may be more practical to determine whether a change in the level of biomarker is an indication of the ineffectiveness of a therapeutic compound (medicament or drug), or, for example, the result of a lapse in patient compliance.

In accordance with the methods of the invention, the first location is suitably a non-clinical location and the second location is a clinical location. In this way, the method provides a means for monitoring and/or measuring treatment efficacy in an individual suffering from a particular disease state, which does not require medical centre visits by the patient and the direct involvement of attendant medical staff/practitioners. Advantageously, in step (b) the transfer is carried out by a public postal service. Hence, the method may be convenient, cost effective, safe and discrete.

Suitably, the biomarker is characteristic of a microorganism against which the therapeutic treatment regime is directed. Thus, in this and other aspects of the invention, the biomarker may be characteristic of a pathogenic microorganism selected from one or more of the group consisting of: bacteria, fungi, viruses, and protozoa. In particular, the methods of the invention are suited for the monitoring of chronic diseases, which may require medium or long-term treatment and regular monitoring of treatment efficacy.

In some embodiments the one or more pathogenic microorganisms include human pathogens that are the causative agents in one or more chronic diseases selected from the group consisting of: sexually transmitted infection, food poisoning, tuberculosis, virally induced cancer, encephalitis, malaria, hepatitis, meningitis, leishmaniasis, African trypanosomiasis, pneumonia, plague, SARS, MRSA, rabies, dysentery, influenza, and typhus.

In some embodiments, the viruses are selected from the group consisting of: cytomegalovirus (CMV), hepatitis A virus (HAV), hepatitis B virus (HBV), hepatitis C virus (HCV), hepatitis E virus (HEV), hepatitis G and GB virus (GBV-C), human immunodeficiency viruses (HIV), human papilloma viruses (HPV), herpes simplex viruses (HSV), Molluscum contagiosum virus (MCV), influenza virus, Epstein-Barr virus (EBV) and varicella-zoster virus (VZV).

In other embodiments, the bacteria are selected from the group consisting of: methicillin-resistant Staphylococcus aureus (MRSA), Clostridium difficile (C. diff), Mycobacterium tuberculosis (TB), Mycoplasma spp., Chlamydia spp., Ureaplasma spp., Neisseria spp., Gardnerella spp., Trichomonas spp., Treponema spp. and Plasmodium spp. More suitably, the bacteria are selected from MRSA, C. diff and TB.

Suitably, the disease is a sexually transmitted infection and the biomarker is associated with one or more microorganisms selected from the group consisting of: Mycoplasma genitalum, Mycoplasma hominis, Chlamydia trachomatis, Ureaplasma urealyticum, Neisseria gonorrhoea, Gardnerella vaginalis, Trichomonas vaginalis, Treponema pallidum, CMV, HAV, HBV, HCV, HEV, GBV-C, HIV-1, HIV-2, HPV, HSV-1, HSV-2, MCV, VZV and EBV.

In one beneficial embodiment of any aspect of the invention, the virus is HIV and the therapeutic compound is an anti-HIV drug.

Advantageously, the biomarker is a nucleic acid molecule and in step (d) the first detection assay comprises a nucleic acid amplification step, wherein the nucleic acid application is directed towards a target sequence of the nucleic acid molecule. Such an assay is convenient for high-throughput procedures and/or automation, so that large numbers of samples and tests can be analysed simultaneously. The target sequence of the nucleic acid molecule suitably comprises at least one highly conserved nucleic acid sequence of the biomarker. In some embodiments, the nucleic acid amplification step may comprise a plurality of pairs of nucleic acid amplification primers that are adapted for amplification of a plurality of target sequences from one or more specified microorganism.

In some embodiments, the biomarker may be a highly conserved polymorphic allele selected from the group consisting of: a single nucleotide polymorphism (SNP), an insertion, a deletion, an inversion, and a substitution. In particular, the highly conserved polymorphic allele may comprise all or a part of a microorganism gene selected from the group consisting of: a bacterial 16S rRNA, a bacterial 32S rRNA, and a viral polymerase.

Beneficially, the solid substrate comprises an absorbent fibrous material, and wherein the solid substrate is comprises one or more reagents that immobilise and inactivate any microorganisms present in the biological sample. Hence, when the biological sample is readily adsorbed into/onto the matrix of the solid substrate and immobilised thereto. Advantageously, any microorganisms that may be present in the sample, and which could be harmful to other individuals are safely contained and inactivated, preventing the risk of contamination of other object and individuals and, therefore, preventing the spread of infection. The solid substrate may comprises a material selected from a cellulose-based paper; a microfibrous membrane; a glass-fibre material; a polymeric fibre material; a nylon material; a silica material; a woven fabric; a non-woven fabric, or a combination thereof.

In this and all other aspects of the invention, the biological sample may be any appropriate fluid or tissue sample obtained from the individual undergoing the treatment regime. For example, the biological sample may comprises at least one of the group consisting of: urine, saliva, blood, sputum, semen, faeces, a nasal swab, tears, a vaginal swab, a rectal swab, a cervical smear, a tissue biopsy, and a urethral swab. Suitably, the biological sample is one that can be readily obtained from the individual, such as urine, saliva, blood and sputum, which the individual may be able to collect from him/herself, without the need for assistance. In some embodiments the biological sample is blood.

In advantageous embodiments, the solid substrate is further capable of immobilising endogenous nucleic acid molecules present in the biological sample, and the method further comprises the step of determining a genotype of the individual based on an endogenous nucleic acid molecule immobilised on the solid substrate. The step of determining a genotype of the individual may suitably comprise: (i) performing a extraction step on the solid substrate to extract endogenous nucleic acid molecules immobilised on and/or within the solid substrate; and (ii) performing a nucleic acid amplification step on the extracted endogenous nucleic acid, wherein the nucleic acid amplification is directed towards at least one target sequence of one or more endogenous gene.

In these embodiments useful genetic data on the individual may be obtained, which may help to reveal reasons and/or explain the result of the assessment of treatment efficacy in the individual concerned. For example, genotype data may provide an explanation for why a particular therapeutic compound is ineffective or less effective than expected; or may indicate more appropriate forms of treatment for the individual.

Accordingly, the one or more endogenous gene is suitably a gene that encodes a protein involved in metabolising the therapeutic compound used in the treatment regime. An advantageous endogenous gene for this purpose is a cytochrome P450 gene.

In accordance with a second aspect of the invention, there is provided a method for determining the concentration of at least a first and a second molecule potentially present in a biological sample obtained from an individual, comprising: (1) depositing the biological sample on a surface, the surface being adapted to immobilise at least the first and the second molecules; (2) extracting the first molecule from the surface and quantifying its concentration; and (3) extracting the second molecule from the surface and quantifying its concentration; wherein the first molecule comprises a nucleic acid and the second molecule comprises a therapeutic compound.

The first molecule is suitably a biomarker, which is characteristic of a pathogenic microorganism against which the second molecule, i.e. the therapeutic compound, is directed. The biomarker suitably comprises a highly conserved polymorphic allele of a pathogenic microorganism as previously discussed.

In embodiments of this aspect of the invention, the pathogenic microorganism may be as already described in relation to the first aspect of the invention. Likewise, the second molecule, i.e. a nucleic acid molecule, is conveniently defined in accordance with the above methods; and the methods for quantifying the nucleic acid in step (2) may be as already described.

In this and other aspects of the invention, the surface is conveniently a solid substrate comprising a material selected from a cellulose-based paper; a microfibrous membrane; a glass-fibre material; a polymeric fibre material; a nylon material; a silica material; a woven fabric; a non-woven fabric, or a combination thereof. Thus, the solid substrate may comprise one or more reagents that immobilise and inactivate any microorganisms that may be present in the biological sample.

Suitably, as in the first aspect of the invention, the nucleic acid immobilised on or in the solid substrate may further comprise endogenous nucleic acid molecules present in the biological sample, and the method may further comprises the step of determining a genotype of the individual based on an endogenous nucleic acid molecule immobilised on the surface. Such a method may comprise: performing a nucleic acid amplification step on the extracted endogenous nucleic acid, wherein the nucleic acid amplification is directed towards at least one target sequence of one or more endogenous gene. The one or more endogenous gene is suitably as described herein.

In a third aspect, the invention also provides a method for determining the drug sensitivity of an individual undergoing a therapeutic treatment regime, comprising measuring the concentration of a therapeutic compound in a biological sample obtained from the individual, and determining a genotype of the individual, the method comprising the steps of: (a) providing at a first location at least one solid substrate, the at least one solid substrate capable of immobilising at least the therapeutic compound and endogenous nucleic acid molecules in the biological sample, and contacting the biological sample with the at least one solid substrate to immobilise the therapeutic compound and the endogenous nucleic acid on and/or within the solid substrate at the first location; (b) transferring the solid substrate with the immobilised therapeutic compound and endogenous nucleic acid molecules to a second location; (c) performing a extraction step on the solid substrate to extract at least the therapeutic compound and the endogenous nucleic acid molecules immobilised on and/or within the solid substrate; and (d) performing a first detection assay to quantify the therapeutic compound and performing a second detection assay on the endogenous nucleic acid molecules to identify a genotype of one or more endogenous gene of the individual.

In one embodiment, the second detection assay includes the step of performing a nucleic acid amplification step on the extracted endogenous nucleic acid, wherein the nucleic acid amplification is directed towards at least one target sequence of one or more endogenous gene, as described in relation to other aspects of the invention. Suitably, at least one of the one or more endogenous genes is an endogenous biomarker of a disease. This aspect of the invention may further comprise detecting and/or quantifying one or more exogenous biomarkers of a disease as described elsewhere. The advantageous features of the solid support and detection methods described in relation to the first and second aspects of the invention are incorporated into the third aspect of the invention, as appropriate.

The invention also provides devices (or kits) for use in monitoring and/or assessing treatment efficacy of a therapeutic regime for treating a disease in an individual.

Accordingly, in a fourth aspect, there is provided a device for use in monitoring a therapeutic treatment regime in an individual having a disease comprising detecting and/or measuring the concentration of a biomarker characteristic of the disease and a therapeutic compound in a biological sample obtained from the individual, the device comprising: a testing surface located within a sealable chamber, the testing surface comprising a solid substrate that is capable of immobilising the biological sample including at least the biomarker and the therapeutic compound either within it or upon its surface; and wherein, in use, once a biological sample has been deposited upon the testing surface, the chamber is sealable around the testing surface such that the biological sample is enclosed within the chamber.

Advantageously, once a biological sample has been sealed inside the chamber, the device is suitable for transporting via a public postal service. Accordingly, the device may be suitable for out-of-clinic (e.g. home), thereby avoiding the need for an individual (patient) to make regular medical centre visits; and beneficially reducing the need for direct involvement of attendant medical staff/practitioners.

The testing surface may be adapted such that, in use, upon deposition of the biological sample on the testing surface any microorganisms that may be present in the biological sample are irreversibly inactivated. Hence, in some embodiments, the solid substrate comprises an absorbent matrix that comprises one or more reagents that immobilise and inactivate any microorganisms present in the biological sample.

Advantageously, the device is adapted such that once it has been sealed, for example, once a biological sample has been deposited on the testing surface, a dedicated tool is required to correctly unseal the chamber. Suitably, the device is disposable, such that once re-opened it may be discarded.

The solid substrate of this aspect of the invention may be the same as in other aspects of the invention. In one embodiment, the solid substrate comprises Whatman FTA® and/or Whatman FTA® elute reagent. This combination of the solid substrate may also be used in the methods of the invention described herein. In some embodiments, the testing surface comprises a solid substrate comprising two or more different materials and/or two or more different reagents that immobilise and inactivate any microorganisms present in the biological sample. For example, the testing surface may comprise a solid substrate comprising two or more different materials, each of the different materials forming a sheet comprising that material, suitably substantially comprising that material, and wherein the two or more sheets are arranged in the form of a stack, one sheet above the other. Alternatively, the testing surface comprises a solid substrate comprising two or more different materials arranged to collectively form a sheet, wherein each of the two or more different materials form segments of the sheet.

Advantageously, the testing surface/solid substrate is suitable for immobilising a biomarker; and/or immobilising a therapeutic compound; and/or immobilising endogenous nucleic acid molecules in the biological sample, as described in relation to the methods of the invention.

Beneficially, upon incubating the solid substrate of the methods and devices in an aqueous solution at a temperature of approximately 70° C. or above, substantially all of the biomarker, therapeutic compound, and any endogenous nucleic acid molecules, where present, are released from the solid substrate into the aqueous solution. Most suitably the solid substrate is incubated in a boiling solution of water or aqueous buffer (e.g. at approximately 100° C.), for example, for 5 or more minutes, such as 10 to 15 minutes. In this way, detection and/or quantification assays can be conveniently carried out.

In further embodiments the invention also provides a therapeutic treatment regime and/or methods for treatment of a disease, which comprises: performing a method of the invention (as previously described), to assess the efficacy of a therapeutic treatment regime; and adjusting the prescribed dose of the therapeutic compound to improve the efficacy of the therapeutic treatment regime; or changing the therapeutic treatment regime to comprise an alternative therapeutic compound. Optionally, a genotype of the individual is also determined.

These and other uses, features and advantages of the invention should be apparent to those skilled in the art from the teachings provided herein.

### DETAILED DESCRIPTION OF THE INVENTION

Prior to setting forth the detailed description of the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs. Where common molecular biology techniques are described it is expected that a person of skill in the art would have knowledge of such techniques, for example from standard texts such as Sambrook J. et al, (2001) Molecular Cloning: a Laboratory Manual, Cold Spring Harbor Press, Cold Spring Harbor, N.Y.

As used herein the phrase “therapeutic treatment regime” refers to an series of approved, predetermined or evolving medical interventions that have the underlying intention of treating, to cure or alleviate, a particular disease (or medical condition) in a patient diagnosed as suffering from that disease (or medical condition). Typically, a therapeutic treatment regime involves a patient undergoing a plurality of medical interventions that are separated in time, and which may be the same or different. Further, a therapeutic treatment regime usually involves the regular monitoring of the patient to assess an improvement or deterioration in the health of the patient over the time period of the treatment regime.

In the context of the present invention, the term “individual” is used to indicate a human or other animal that is undergoing a medical treatment, i.e. a therapeutic treatment regime. In some circumstances, i.e. where the individual has been previously diagnosed as having a disease for which that individual is undergoing a therapeutic treatment regime, then the term “individual” can be considered to be synonymous with the word “patient”. However, the term individual is used to also encompass individuals whom are undergoing a therapeutic treatment regime but whom have not been diagnosed with the particular disease to which the therapeutic treatment regime is intended to treat. For example, such an individual may be involved in a medical or clinical trial or a diagnostic testing. As noted, an individual may be a human subject whom is undergoing treatment for a disease or involved in a clinical trial; or an animal that is undergoing treatment for a disease or is being used in a clinical trial. Thus, the methods and devices of the invention may be for veterinary use.

By the word “biomarker” it is meant a biological entity, for example, a compound, molecule, or part of a compound or molecule, which is indicative of a particular disease and which can be used to characterise the disease or, in general terms, the type of the disease. In particular, a biomarker may be a protein, a peptide or polypeptide sequence, a (poly)nucleic acid (DNA or RNA) or a fragment of a nucleic acid.

A “therapeutic compound”, as used herein, is a compound that is intended to treat (either to cure or alleviate) the symptoms of a disease. Thus, the term encompasses a drug, pharmaceutical molecule/compound, medicament, medicine and the like.

The term “biological sample” as used herein is intended to encompass an amount of a biological material that has been obtained from an individual, which may be a human or a non-human animal. The sample may be liquid or solid and can suitably include, for example: urine; faeces; vaginal, nasal, rectal or oral swabs; blood, saliva and/or sputum; semen; vaginal or urethral discharges and swabs thereof; tears (i.e. lacrimal secretions); biopsy tissue samples; and swabs of surfaces upon which any of the aforementioned secretions and substances may have been deposited. Whilst the biological sample may contain cells, tissue and/or DNA originating from a host organism, e.g. a human patient, it may also contain microorganisms present within that host.

By “immobilise” it is meant that the immobilised entity (e.g. molecule or organism) is fixed, adsorbed or otherwise attached to the relevant surface or solid substrate such that it cannot become detached therefrom, without external intervention. Thus, where the immobilised entity is a molecule it cannot become detached by diffusion, for example; and where the immobilised entity is an organism it cannot become detached under its own propulsion. Once immobilised, the entity may be removed (or disattached) from the surface or solid substrate by extraction, for example, by an extraction step or procedure. The extraction step or procedure depends on the type of entity that is immobilised and the surface or solid substrate to which it is immobilised. Suitable extraction steps or procedures are known to the person skilled in the art and include, for example, boiling in an aqueous solution. Suitably, where the immobilised entity is an organism, the act of immobilisation further inactivates the organism or otherwise renders it unviable, such that even following disattachment the organism is unable to grow, multiply, reproduce or infect another organism.

The phrase “disease state” is intended to be used qualitatively and refers generally to the level or condition or extent of the particular disease in an individual. Broadly speaking, the disease state can be considered to represent the progress of the disease. Thus, the disease state may simply be considered to be “good” when the level of infection or extent of the disease is significantly lower than expected; the disease state may be “fair” (or “normal”) when the level of infection or extent of the disease is approximately the same as expected; and the disease state may be “bad” when the level of infection or extent of the disease is significantly worse than expected. Purely by way of example, in a patient infected with HIV, one disease state may broadly relate to blood HIV concentration levels of less than 50 copies of the virus per ml of blood; a different disease state may be considered to apply when the blood HIV concentration levels is between 50 and 500 copies of the virus per ml of blood; and a different disease state may be considered to apply when blood HIV concentration levels are higher than 500 copies of the virus per ml of blood.

The “efficacy of the therapeutic treatment regime” is related to the disease state of the individual in the sense that the therapeutic treatment regime is considered to be efficacious (or effective) if the progress of the disease (or the disease state) is as expected or better than would be expected under that therapeutic treatment regime; whereas the therapeutic treatment regime should be considered to be non-efficacious (or ineffective) if the disease state is worse than should be expected under the particular therapeutic treatment regime.

By “target level” in the context of the target level of a therapeutic compound, it is meant the amount or concentration of the particular therapeutic compound that is considered to be the recommended therapeutically effective amount of that therapeutic compound for treatment of the relevant disease in the appropriate biological sample. Thus, the target level will of course be dependent on the relevant disease, the therapeutic compound concerned and perhaps the individual (e.g. male/female). Depending on the disease and the therapeutic compound, the target level may be a specific amount/concentration, or a concentration/amount range. Typically, the target level should be considered to represent the manufacturers' recommended level or the level widely acknowledged to be the target level by the skilled practitioners in the appropriate medical field.

By “non-clinical” in the sense of a non-clinical location, it is meant that the location or building is not (or is not intended to be) a medical centre, hospital, clinic or medical research laboratory or centre. Moreover, by non-clinical it is meant that the presence of a clinician (e.g. a medically trained person, such as a medic, doctor, nurse, or other therapist including a veterinary practitioner) is not necessary. By contrast, by “clinical” in the sense of a clinical location, it is meant a hospital, clinic, surgery, medical research laboratory or other location where a clinician would be expected to be present, or where a clinician would perform the relevant task.

The phrase “public postal service” refers to a national, state or privately run postal service that does not, under normal circumstances and without taking special precautions, handle and/or deliver hazardous materials or packages, such as those containing potentially infectious agents. Thus, the phrase public postal service is also intended to imply that special precautions for the handling and/or delivery of the item concerned are not required to meet any national, state or private legal requirements or recommendations in the country concerned.

The term “microorganism(s)” as used herein is intended to denote one or more species of microorganism that may or may not be present in a biological sample. The microorganisms are suitably of viral, bacterial, fungal (including unicellular yeast) and/or protozoan (including plasmodium) origin. Typically, the specified microorganisms will be pathogenic to an animal host at some point in their life cycle. However, the present invention may detect species of microorganisms that exhibit dormancy, commensal infection or sub-clinical infection. The word “pathogenic” is intended to have its normal definition in the art, for example, an agent (or organism) causing or capable of causing disease.

The term “chronic” is intended to take its usual meaning, for example, in the sense of a chronic disease, the disease is of a marked by long duration, or may display frequent recurrence. Likewise, the term “acute” takes its normal meaning of having a sudden onset, and/or sharp rise, and/or short course.

A “nucleic acid” or a “nucleic acid sequence” is a single or double stranded covalently-linked sequence of nucleotides in which the 3′ and 5′ ends on each nucleotide are typically joined by phosphodiester bonds. The nucleic acid sequences are typically polynucleotides that may be made up of deoxyribonucleotide bases or ribonucleotide bases, and may, therefore, include DNA, RNA and DNA/RNA hybrid molecules. The phrase “nucleic acid molecule” is used herein interchangeably with the phrase “polynucleotide molecule. A nucleic acid molecule may be manufactured synthetically in vitro or isolated from natural sources. Sizes of nucleic acid sequences are typically expressed as the number of base pairs (bp) for double stranded polynucleotides, or in the case of single stranded polynucleotides as the number of nucleotides (nt). One thousand by or nt is equal to a kilobase (kb). Polynucleotides of less than around 40 nucleotides in length are typically called “oligonucleotides”. By the term “primer” it is meant a relatively short single stranded polynucleotide or oligonucleotide that may be used, for example, in a nucleic acid amplification step (or a primer extension reaction/step). Typically, a primer is less than 120 bases (or nt), suitably less than 80 bases, more suitably less than 60 bases, and most suitably a primer is less than 40 bases in length, such as an oligonucleotide.

A “target sequence” of a nucleic acid molecule is a region of a single or double stranded polynucleotide that is intended to be analysed by subjecting it to a nucleic acid amplification step or a primer extension step, in order to determine its presence and/or its concentration. Typically, a target sequence is selected on the basis that it is unique for a particular individual or microorganism or for a sub-group or type of individual or microorganism.

The term “nucleic acid amplification reaction” (or step) as used herein denotes any of a number of related enzymatic techniques that utilise a DNA polymerase to amplify a specified sequence of DNA using serial rounds of primer extension, denaturation and hybridisation. Typically the DNA polymerase is thermostable, and the polymerase chain reaction (PCR) is the preferred nucleic acid amplification reaction used in the methods of the present invention. In a nucleic acid amplification reaction a pair of primers hybridise at either end (i.e. the 5′ and 3′ ends) of the target nucleic acid sequence. The primer than hybridises to the 5′ end of the target sequence may be called the forward primer and it has a sequence complementary to the coding (or top) strand of the (double stranded) DNA molecule; and the primer than hybridises to the 3′ end of the target sequence may be called the reverse primer and it has a sequence complementary to the non-coding (or bottom) strand of the (double stranded) DNA molecule.

The term “primer extension reaction” is intended to denote a reaction in which nucleic acid primers are designed to hybridise with a target sequence and be enzymatically extended by adding one or more nucleotides to the 3′-end of the primer. The primers hybridise at a position on a given target sequence that is immediately 5′ to or a few bases upstream of the sequence to be extended, such as a sequence containing a polymorphism (e.g. a single nucleotide polymorphism). In embodiments of the invention where the primer hybridises on bases upstream of a known SNP site, single-base primer-extension acts on primer-target sequence hybrids to add a single chain-terminating nucleotide, often a dideoxynucleotide. The only one of four nucleotides that will extend the primer is the one that is complementary to the sequence on the target strand. The identity of the added nucleotide is determined during the analysis phase of the method of the invention, described in more detail below.

The term “polymorphic allele” is used herein to denote any two or more alternative forms of genetic sequence occupying the same chromosomal locus and controlling the same inherited characteristic. Allelic variation arises naturally though mutation, and may result in phenotypic polymorphism within populations or may result in a conservative (non-phenotypic) polymorphism. Gene mutations typically result in an altered nucleic acid sequence. As used herein, the phenomenon of allelic polymorphism is utilised in respect of single nucleotide polymorphisms (SNPs), insertions, deletions, inversions and substitutions, all of which can occur even in genes that are highly conserved in a given species. SNPs are polymorphisms where the alleles differ by the replacement/substitution of a single nucleotide in the DNA sequence at a given position in the genome. In highly conserved genes, such as 16S rRNA in bacteria, SNPs are highly species and strain specific, thereby allowing accurate genotyping information to be obtained. Other highly conserved regions in microorganisms include the bacterial 32S rRNA gene, yeast 16S and 18S rRNA genes and viral polymerase genes. Nevertheless, it is within the remit of the skilled person to utilise bioinformatics techniques to identify SNPs in other alternative conserved regions of the genome for a given microorganism. Polymorphisms, such as those described above, may be linked to specific phenotypic traits in the organism under test. For instance, antibiotic resistance is associated with mutation and, thus, polymorphism. Nevertheless, the method of the present invention is not restricted to identification of only polymorphic positions in the genomes of the microorganisms under test. Where competitor control sequences are used for a given conserved target sequence, the term “polymorphic allele” is used loosely to denote the variation at a given part of the sequence between the wild-type sequence (that being tested for) and the artificial competitor sequence. In this instance it will be appreciated that the so-called polymorphism is simply to assist in differentiation of the reaction products of primer extension on the competitor template versus the wild type template, as the respective reaction products will have differing relative molecular masses.

By “endogenous” it is meant that the entity concerned, for example, a nucleic acid, is derived from the individual from which the biological sample is derived. Therefore, an endogenous nucleic acid or gene is a nucleic acid or gene of human or non-human mammalian origin. In the context of the invention, a non-endogenous (i.e. an exogenous) nucleic acid or other entity is derived from a microorganism that has infected the individual (or it could be from contamination in the biological sample or in the test).

By “genotype” in the context of “determining a genotype of an individual” it is meant the genetic identity/sequence/composition at a specific gene locus or set of loci of the relevant individual. Of particular interest in the context of the invention is genetic identity of alleles or different forms of a target gene or gene sequence in the individual, which may be used to explain or predict a characteristic of the individual, for example, the way in which that individual may react to, or the rate at which that individual may metabolise a particular therapeutic compound.

### Monitoring a Therapeutic Treatment Regime

There are many diseases of the human or animal body that can be monitored by measuring one or more biomarkers of that disease in a biological sample obtained from an individual suffering from the disease. Where the disease is essentially host-mediated, such as a cancer (which is not caused by a viral infection), an autoimmune disease, or any other disease of the human or animal body that is not caused by a pathogenic organism, then the biomarker of the disease may be host-related (or endogenous). By contrast, where the disease is caused by a pathogenic organism, then the biomarker may be associated with that pathogen (i.e. it is of non-host or exogenous origin). Hence, a suitable biomarker of a disease may be any biological entity that can be measured or detected: such as a cell-surface expressed antigen, an antibody, enzyme or other protein molecule characteristic of a particular disease (or medical condition); or it may be a characteristic gene (or allele) or gene sequence of a microorganism, such as a virus, bacterium, fungus or plasmodium.

By detecting and/or quantifying the relevant biomarker, the extent, progression or reoccurrence of the disease (or medical condition) may be monitored. The monitoring of disease progression is particularly relevant for chronic diseases, where the disease may exist in and affect the afflicted individual for a long period of time or even for life, as with malaria and HIV. The treatment of chronic diseases may require careful management, because there is a risk that a patient could be unresponsive to the standard prescribed medicament (therapeutic compound) and without active monitoring the unresponsiveness may go unchecked for a long period of time. Alternatively, the patient could become unresponsive to the medication, for example, because the disease being treated (in the case of pathogenic diseases) might develop immunity over prolonged exposure. In other cases, a patient him/herself may develop an immune response to the drug, leading to faster clearance of the drug from the system. It is even possible that other conditions or factors in the life of the individual could start to influence the effectiveness of the medicament by, for example, increasing the rate of clearance of the therapeutic compound from the body, such as by metabolism.

Therefore, it is known in the treatment of chronic disease for a patient receiving a therapeutic treatment regime to undergo fairly regular (e.g. every 1 to 6 months) medical check-ups, to assess disease progression (or disease state). By way of example, a patient suffering from a cancer may be assessed every 3 to 6 months, while a patient being treated for HIV infection may be tested every 6 to 8 weeks. At each check-up there is a level of the disease that the medical practitioner or other health worker may consider to be acceptable under the circumstance. For instance, in HIV therapy, the target may be to keep the concentration of HIV in the patient's blood or plasma to below 50 copies of the virus per ml of fluid.

However, a problem arises when for some reason the disease state is worse than was expected. Typically, in the art the answer might be to either: increase the dose of the existing therapy; or change to an alternative therapy, if there is one available. Each of these “solutions” can involve considerably risks. In this regard, by increasing the dose of a therapeutic compound there is the risk of greatly enhancing any potential toxic side effects of the compound, and in any case, the treatment may remain ineffective and encourage the development of strong immunity where the disease involves a pathogenic organism. By changing drugs, there is a risk of triggering new adverse side-effects in the individual, or allowing a microorganism to develop additional immunity. Moreover, neither of these responses to the lack of therapeutic success takes account of the actual therapeutically effective concentration range of the respective therapeutic compound.

Therapeutic drug monitoring (TDM) involves the measurement of the amount of a drug (or therapeutic compound) in a body fluid of a patient (e.g. blood, plasma or saliva). By taking a measurement of drug concentration in an appropriate biological sample of the patient, and comparing that measurement to the pharmacologically recommended therapeutically effective range (or target level/range), it can be seen whether or not a drug has been administered in the correct quantity for optimal treatment of that disease. If it is found that the measured concentration of drug is below the target level, this may indicate that the medication is not providing its optimal therapeutic effect. If however the drug concentration is too high, then there may be potential toxic side effects. This approach can be particularly important if a drug is known to have a narrow effective range.

However, the concentration of a drug in a patient's body can fluctuate significantly in view of a number of different and common factors. For example, the concentration may depend on the time since the last administration of the drug; whether the previous dose was taken with or without food; the general health of the patient; and even whether the patient has been taking the treatments in the correct doses or time intervals. Therefore, TDM also has a number of important limitations. TDM in isolation also does not take account of another important aspect of disease management/treatment, which is the control over the bioavailable time-course of a drug. In some circumstances it may be the peak drug level (i.e. the maximum bioavailable amount of a drug) that is therapeutically effective; whereas in other cases it may be the steady-state or trough levels of the drug that are important for treatment.

The present invention is, therefore, based in part on the recognition that individuals may absorb, process (e.g. metabolise) and/or eliminate drugs at significantly differing rates. Hence, levels of a particular drug in a biological sample obtained from a patient (e.g. blood) may vary considerably amongst patients who have taken exactly the same dose of medication, even where the patients took the drug at exactly the same time or under the same conditions.

Thus, the invention provides methods and devices (or kits) that combine the benefits of TDM (i.e. the monitoring of drug concentration levels in a biological sample of an individual), with a measure or indication of the level of disease (or disease state) in the individual undergoing treatment for that disease. Conveniently, the disease state of the individual can be monitored, measured, or estimated by detecting a particular biomarker in the biological sample of the individual that is associated with the disease state of interest. Accordingly, the effectiveness of the treatment regime in that individual can be assessed through the combination of both disease response and patient response to the drug. Further, in the case of an ineffective treatment regime, informed conclusions may then be drawn on the reasons for the lack of success, and safe changes to the therapeutic treatment regime may be implemented.

The invention is also based on the recognition that standard procedures for the regular monitoring/assessment of patients being treated for chronic diseases in particular can be very expensive, both financially and in terms of medical resources. In this regard, it is thought that it may cost as much as $1000 per appointment (depending on the disease) for an outpatient to be assessed by a medical practitioner and for the relevant test results to be obtained. Furthermore, it is clear that much of this expense is due to the use of medical resources and time in attending an appointment with a medical practitioner to have a biological sample taken; for tests to be carried out on the sample and the results to be reported to a medical practitioner; and for the patient to again attend an appointment with the medical practitioner to receive the results; not to mention the time and costs of missed appointments. Apart from the obvious expense of such procedures, there should also be considered the significant drain on medical resources/services.

Thus, the invention provides methods and devices (kits) that are suitable for use in different locations, for example, in two locations, such as in a non-clinical setting or location and in a clinical location as required. Accordingly, at least a part of the prior art routines for the monitoring of an outpatient undergoing a therapeutic treatment regime is obviated, with significant economic and social benefits. In particular, the invention allows the initial stage, at least, in the monitoring of a therapeutic treatment regime to be carried out at a first location without the need for a medical practitioner (or other health worker) to be present. A further advantage is achieved by the methods and devices of the invention by enabling at least a part of the monitoring process to be carried out in a non-clinical location, thus avoiding the need for an individual to attend a medical centre (or similar). More advantageously, the invention relates to methods and devices that allow the biological sample from the patient or individual to be obtained/provided at a non-clinical location in the absence of a medical practitioner. An additional benefit of the methods of the invention is that by not requiring the attendance of an individual at a medical centre for the taking of a biological sample, the process of monitoring a patient can be far more discrete that is currently possible, providing important social and personal benefits to the patient. In point of fact, unless the tests on a patient's biological sample indicate a medical need to consult with a medical practitioner, the patient need not attend a medical centre, and thereby, avoid the risks of exposing him/herself to further infections, or of being identified.

Another benefit of the methods and devices of the invention is achieved by providing a system that does not even require the use of medical centre employees to transfer a biological sample that has been obtained from an individual, from the place at which it was obtained to the place at which it is to be tested. In this regard, while the method of the invention involves obtaining a biological sample at a first location, and testing the biological sample at a second location; the device of the invention is suitable for the safe transfer of the biological sample from the first to the second location. More advantageously, the device is suitable for transportation, handling and delivery by a public postal service, without the need to special handling precautions. This further benefits the patient in terms of financial costs and anonymity.

### Diseases

Embodiments of the invention may be application to all types of diseases, whether they are thought to be caused by pathogenic organisms, or are due to internal factors in the individual, such as genetic mutation/predisposition or other environmental triggers. The important factor, however, being that there is a biomarker that is indicative of the particular disease (or type of disease), and which biomarker can be obtained from a suitable biological sample in an afflicted individual.

In particular, the invention relates to chronic diseases that may require long-term therapeutic treatment regimes, and especially those that require (or would benefit from) regular monitoring.

Relevant pathogenic organisms include microorganisms such as: bacteria, fungi, viruses, and protozoa. More specifically, the human pathogens that cause sexually transmitted infection (STI), tuberculosis, virally induced cancer, encephalitis, malaria, hepatitis, meningitis, leishmaniasis, African trypanosomiasis, pneumonia, plague, SARS, MRSA, rabies, dysentery, influenza, and typhus. Where the disease is an STI, suitable pathogenic microorganisms may be Mycoplasma genitalum, Mycoplasma hominis, Chlamydia trachomatis, Ureaplasma urealyticum, Neisseria gonorrhoea, Gardnerella vaginalis, Trichomonas vaginalis, Treponema pallidum, CMV, HAV, HBV, HCV, HEV, GBV-C, HIV-1, HIV-2, HPV, HSV-1, HSV-2, MCV, VZV and EBV.

Relevant bacteria include species such as: methicillin-resistant Staphylococcus aureus (MRSA), Clostridium difficile (C. diff), Mycobacterium tuberculosis (TB), Mycoplasma spp., Chlamydia spp., Ureaplasma spp., Neisseria spp., Gardnerella spp., Trichomonas spp., Treponema spp. and Plasmodium spp. Particularly relevant are MRSA, C. diff and TB.

Viruses that may be subject to therapeutic treatment regimes include: cytomegalovirus (CMV), hepatitis A virus (HAV), hepatitis B virus (HBV), hepatitis C virus (HCV), hepatitis E virus (HEV), hepatitis G and GB virus (GBV-C), human immunodeficiency viruses (HIV), human papilloma viruses (HPV), herpes simplex viruses (HSV), Molluscum contagiosum virus (MCV), influenza virus, Epstein-Barr virus (EBV) and varicella-zoster virus (VZV). Any virus that may be associated with a chronic disease may, however, be monitored using the methods of the invention. A particularly suitable virus is HIV.

Diseases that may be caused other than from pathogenic microorganisms include, inter alia, cancer, autoimmune disease (e.g. multiple sclerosis (MS)), neurodegenerative disease (e.g. MS, motor neuron disease), and dementia (e.g. Alzheimer's disease).

### Methods

The methods of the invention require that a biological sample is obtained from an individual or a patient, whether human or non-human animal.

The invention is applicable for a wide range of biological samples, for example, the sample may comprise: urine; faeces; vaginal, nasal, rectal or oral swabs; blood, saliva and/or sputum; semen; vaginal or urethral discharges and swabs thereof; tears (i.e. lacrimal secretions); biopsy tissue; and swabs of surfaces upon which any such secretions and/or substances may have been deposited. The biological sample may, therefore, be a solid or a liquid: although suitably it comprises a liquid. More suitably, the biological sample is readily obtained without the need for a trained or medically qualified practitioner, such as a doctor or nurse. Advantageously, therefore, the biological sample comprises nasal or oral swabs; blood, saliva and/or sputum, and tears (i.e. lacrimal secretions). More suitably, the biological sample is an oral swabs, blood or saliva; and most suitably, blood.

Beneficially, where a disease is caused by a pathogenic microorganism, the biological sample may contain cells, tissue and/or DNA originating from the host individual (e.g. a human patient) as well as microorganisms present within that individual.

The methods of the invention relate to the deposition of the biological sample on (or the contacting of the sample with) a solid substrate, surface (or testing surface), or solid support; for transportation of the sample from a first to a second location. In relation to the methods of the invention, it is important that the solid substrate is capable of immobilising at least the biomarker of the disease (whether that is endogenous or exogenous); the therapeutic compound being used in the therapeutic treatment regime to treat the disease (or being that is being used in a clinical trial, for example); and, where the method involves genotyping of the individual, endogenous nucleic acids. Accordingly, the solid substrate (or surface) of the invention advantageously has the ability to immobilise more than one type of molecule at the same time: for example, nucleic acids and/or polypeptides and/or small molecule pharmaceutical compounds. In one embodiment the solid substrate immobilises at least a first molecule and a second molecule; and advantageously, the first molecule comprises a nucleic acid molecule, and the second molecule is a therapeutic compound. The nucleic acid molecule and/or the peptide molecule may be host derived, pathogen derived or, typically, both.

It is particularly beneficial for the performance of the invention that the transportation of the biological sample does not expose other people or animals to possible infection. Therefore, in advantageous embodiments, the solid substrate is further capable of immobilising and inactivating any microorganisms that may be present in the biological sample.

With the above in mind, the solid substrate of the invention is suitably selected from an absorbent fibrous material impregnated with one or more reagents that act to immobilise and inactivate any microorganisms present in the biological sample; or, even more simply, to cause immobilisation of nucleic acids contained within the microorganisms itself. Such reagents may include detergent compounds: anionic or cationic detergents, chelating agents (e.g. EDTA), urea and/or uric acid. The solid substrate itself may incorporate as an absorbent a material selected from: a cellulose-based paper (e.g. blotting paper); a microfibrous membrane; a glass-fibre material; a polymeric fibre material (e.g. a nylon filter membrane); a woven fabric; and a non-woven fabric. Suitable solid substrates are described, for example, in U.S. Pat. Nos. 5,496,562, 5,756,126, 5,807,527, 5,939,259, 5,972,386, 5,985,327, 6,168,922, 6,746,841 and 6,750,059. In specific embodiments of the present invention the solid substrate includes filter paper treated with Whatman FTA® or Whatman FTA Elute® reagent, such as Whatman ETA® paper or Whatman ETA® Elute paper.

Other systems for adsorption of biological materials, such as nucleic acids and polypeptides to a solid substrate are known to the person skilled in the art, and may be incorporated into the methods and devices as necessary. In this regard, the need to immobilise small molecules: such as therapeutic compounds that may be used for the treatment of diseases such as viral infections, and antibiotics, the solid substrate may be treated with suitable reagents that attract these molecules. Charge-attraction and covalent linkage techniques and reagents may be beneficial in this respect, as they may be for immobilising polypeptides and nucleic acids.

Charge attraction is a beneficial option because the immobilised molecules may be eluted from the solid substrate or surface by elution with a suitable buffer, for example, which may not destroy the chemical identity of the compound. In this way, it may also be possible to readily separate the therapeutic compound from the biomarker (e.g. polypeptide or nucleic acid) for ease of downstream detection and/or quantification. Charge attraction may be achieved either through modification of the membrane surface during manufacture or by post-production coating with a charged species, such as poly-L-lysine. By way of non-limiting example, this process has been demonstrated successfully with solid substrates including polyvinylidene fluoride (PVDF) or nylon membranes and also glass slides. Other methods that are known to the person skilled in the art may also be used.

Covalent linkage is an increasingly popular technique for immobilising nucleic acids onto a range of surfaces, although it may also be used for immobilising polypeptides and/or therapeutic compounds as already noted. There are a number of journals whose focus is the attachment of nucleic acids to solid surfaces via traditional chemistry. There are a number of linking chemistries that can be used, with the most common being amine, carbonyl, carboxyl, and thiol. Covalent linkage can be used advantageously to control the orientation or point of attachment of the linked species (e.g. nucleic acid molecule), for example, which may be useful where nucleic acid analysis is to be performed without extracting the nucleic acid or polypeptide from the solid substrate. It should be noted that in some embodiments, the process of extracting the first or the second molecule is optional. Thus, the invention further relates to a method a described, wherein step (c) is optional.

This immobilization technique can be most efficient when the reaction takes place through the end groups. The mode of attachment can be selected according to requirements, from simple linkages, such as a Schiffs base reaction (where an aldehyde group reacts with an amine followed by reduction), to more complex systems using specially designed linkers.

A controlled-chemistry linkage makes possible controlled attachment of a biomarker or therapeutic compound via a terminal group. Further, it may be desirable to introduce a spacer of a particular size to ensure that the immobilised molecule can move freely to hybridise with any added probe.

A significant advantage of the methods (and devices) of the present invention is that a solid substrate treated with a reagent, such as the Whatman FTA Elute® reagent, can be used by an individual in a non-clinical setting. For instance, it is envisaged that the initial biological sample collection phase of the method of the invention could be carried out by an individual purchasing a device or kit of the invention and simply wetting the solid substrate with, say, a sample of urine, saliva or blood. The solid substrate will immobilise any microorganisms present in the biological sample, such that infectious pathogens are safely rendered non-infectious and the sample can be transmitted to a testing location without the need for expensive handling (i.e. refrigeration, or additional chemical fixing), for instance by regular public postal services.

The immobilised microorganism DNA can be easily extracted from the solid substrate in the testing location, typically by using a simple heat and water elution step. For example, by boiling the solid substrate (plus sample) in water of buffer at 100° C. for 10 to 15 minutes. The invention, thus, provides a system in which biological sample collection can even be performed at home or in the field. In some embodiment, samples may be stored indefinitely and then tested at a later date. The benefits of this system are significant as to-date most diagnostic testing is hampered by the need for sample collection to be performed in a clinical environment, which greatly reduces uptake amongst the population as a whole.

In some embodiments, in may be desirable to calculate or at least estimate the concentration of a molecule (such as a biomarker of a pathogen and/or a therapeutic compound), in the biological sample. Any suitable means for calculating the concentration of the relevant molecule can be used. In one embodiment, the sample size or volume is controlled, predetermined, measured, or otherwise known. To control the sample size it may be possible to load the biological sample onto a solid substrate or surface that is only capable of immobilising or adsorbing a particular amount or volume of the sample. By way of non-limiting example, the solid substrate may be sized or treated to be able to only adsorb 50 μl of a liquid, such as blood, urine or saliva. In this way, even if the solid substrate were saturated, then only a specific volume of sample would be collected. In another embodiment, the sample may be collected using a collection device that is only capable of collecting or extracting a specific, predetermined amount or volume of a biological sample, for example, 50 μl; and then transferring that sample to a solid substrate, such as a blotting paper. Suitable means for controlling the size of the biological sample collect include a capillary having a defined internal volume, and a loop that can hold a defined volume of liquid or material within it. In another embodiment, it may be assumed that a drop of sample placed onto a solid substrate or surface typically has a volume of approximately 50 μl. Such an estimation of sample size may be sufficiently accurate for calculating the concentration of a molecule, particularly where high variability in the concentration of the molecule is possible: for instance, in the case of viral load in HIV infected patients.

In alternative embodiments, the concentration of a particular molecule, particularly of a nucleic acid molecule, may be determined by using genomic DNA as a concentration marker; or by spiking the sample with a known number of copies of a suitably foreign (exogenous) nucleic acid, such as jellyfish or plant DNA, which can then be used as a concentration marker. In the case of a genomic DNA marker, a nucleic acid amplification reaction of a genomic DNA marker, such as 16S rRNA (or any alternative means known to the skilled person), may be used to calculate the concentration of the genomic DNA in the biological sample. In this way, it may then be possible to calculate the number of host cells in the biological sample, and thus, the volume of the sample. This technique may be particularly suitable when the biological sample is blood, and the concentration of host cells in blood (e.g. white and red blood cells) can be estimated or measured.

It is particularly advantageous to calculate the concentration of the therapeutic drug in the biological sample for measurement of TDM, so that it is possible to determine how rapidly the compound is metabolised by the individual concerned. For TDM measurements the biological sample is most suitably blood.

In other cases, for example, where the biomarker is an endogenous nucleic acid, it may simply be sufficient to detect the molecule, rather than to quantify it or calculate its concentration.

### Devices and Kits

The invention further relates to testing devices and kits for use monitoring a therapeutic treatment regime, and generally, for use in the methods of the invention.

Advantageously, the device of the invention comprises a testing surface or solid substrate capable of immobilising a biological sample: particularly, a biomarker and a therapeutic compound within the biological sample; and a chamber suitable for enclosing the testing surface or solid substrate with means for sealing the testing surface or solid substrate within a chamber. In this way, users and handlers of the device are shielded from the biological sample except when the device is opened by unsealing the chamber.

In order to immobilise at least a first and a second molecule from the biological sample, such as a biomarker of the disease and a therapeutic compound intended to treat the disease, the testing surface or solid substrate may be in the form described above.

The testing surface may be further adapted where it is beneficial to provide a different surface or solid substrate for the immobilisation of each of the molecules that are to be immobilised. For example, one type of surface (for instance, a charge-attraction surface), may be selected or optimal for the immobilisation of a particular therapeutic molecule or molecules, while a different type of surface (such as a covalent-linkage surface), may be most suitable for immobilisation of the selected biomarker, for example, a nucleic acid molecule (e.g. RNA). In these embodiments, the surface may be adapted using any suitable method. Thus, one or more solid substrates may be combined with one or more additional reagents.

In one embodiment, a single solid substrate, for example, a microfibrous membrane, may be impregnated at the same or different locations with two or more different reagents, in order to generate the capacity to immobilise molecules by two or more different means, respectively. For instance, different sections of the membrane may be impregnated with the two or more different reagents so that different zones (regions or areas) of the membrane are predetermined to immobilise (and adsorb) predetermined different molecules—for example, the biomarker may be immobilised in one zone and the therapeutic compound may be immobilised in a different zone of the membrane.

In another embodiment, two or more different types of solid substrate are combined to create a testing surface, such as a cellulose-based paper and a glass-fibre material. The two or more different solid substrates may be formed into individual layers/membranes and arranged in, for example, a stack or sandwich structure within the device. The two or more different solid substrates may further be impregnated with one or more reagents to create predetermined adsorbing/binding capacities to immobilise molecules. In this way, in use, a biological sample, such as a drop of blood, may be deposited on the top layer of the testing surface and it may then be adsorbed into and through the top layer and into the one or more lower layers of the testing surface. By way of example, the first or second molecule (for example, a nucleic acid or polypeptide biomarker) may be immobilised in the top layer, while the therapeutic compound may be immobilised in the second layer/solid substrate. When the sample is to be analysed, it may then be a relatively simple process to separate the layers and detect the first and second molecules separately.

In an alternative embodiment, the two or more different solid substrates may be made in the form of a single sheet, layer or membrane; for instance, by combining the materials before formation of the membrane, or by forming individual membranes and then joining the different materials together. In one embodiment, segments of membranes of solid substrates are combined (e.g. in semi circles or segments) to form a single layered membrane having different compositions in different zones. Thereafter, the two or more different solid substrates may further be impregnated with the same or different reagents to create predetermined adsorbing/binding capacities to immobilise particular molecules in different regions of the testing surface.

Suitably, the device is suitable for transportation by a public delivery service, and particularly, by a public postal service, without requiring special safety precautions, as previously discussed. The suitability for such transportation and delivery may be due to the means by which the biological sample is immobilised and pathogens are inactivated, or by the means by which the device is sealed to contain the biological sample, or by a combination of both of these factors.

Advantageously, the means of sealing the chamber is “tamper-proof”, in the sense that it can not be correctly opened (i.e. without damaging the chamber), without the use of a specialised or dedicated opening tool. It will be appreciated that may objects can be opened in an unauthorised, incorrect manner, for example by breaking the device; and breaking (i.e. forcing open) the device or kit does not imply that the device is not tamper-proof, or that it can be correctly opened without the use of a specialised tool. Beneficially, the specialised or dedicated opening tool is only possessed by a skilled/authorised person or technician, whom is responsible for the testing or handling of the biological sample. The specialised or dedicated opening tool is suitably in the form or a key. It should be appreciated, however, than in alternative embodiments, the device is does not comprise a dedicated opening tool, and in this case it may be intended that the device is opened (by an authorised person) by breaking it open.

Beneficially, the device or kit includes instructions for use, for example, which direct the user (individual) to place a biological sample—such as a drop of blood—on the testing surface. The testing surface comprises a solid substrate of the type described above. After the sample is deposited on the testing surface the sealable chamber can be closed such that it encapsulates and protects the testing surface from further interference. In the sealed state the device remains secure from outside interference or contamination and can be despatched to a testing facility located remotely from the user's home. At the testing facility the sealable chamber can be opened (if necessary by breaking the chamber open) allowing access to the testing surface for analytical purposes according to the method of the invention.

### Analytical Procedures

Having obtained a testing surface or solid substrate having a biological sample immobilised on or in it, the first and second molecule, for example, the biomarker and therapeutic compound are advantageously extracted from the surface or support. The means of extraction may include (as previously described), elution using a suitable elution solution or buffer, and/or heat treatment such as boiling in the solution or buffer. In other circumstances, extraction may be by enzymatic or other chemical reaction, for instance UV light to break chemical bonds. Suitable means for extraction are known to the person skilled in the art.

It is then necessary to detect and/or quantify the respective molecules, which can be done in any suitable manner.

Techniques for the detection and/or quantification of nucleic acids in a sample include but are not limited to: RT-PCR, ligase chain reaction, RNase protection assay, and northern blot assay (Sambrook et al., Molecular Cloning: a Laboratory Manual, Cold Spring Harbor Press, Cold Spring Harbor, N.Y.). In addition, suitable probes may be immobilised on a substrate so as to form part of an array or microarray, that allows screening of any nucleic acid molecules obtained from the sample for many different biomarkers. In fact, any hybridisation or detection assay may be used, and particularly those that give either an amplified signal from a probe (for example), or an amplified product, as described below.

A preferred means for quantifying a nucleic acid is to use a nucleic acid amplification step, for example PCR, or RT-PCR (e.g. real-time quantitative PCR) in the case that the nucleic acid is RNA (for example, the HIV genome). Suitably, the nucleic acid amplification is performed using primer pairs that target unique nucleic acid sequences in the target molecule or biomarker. Both PCR and RT-PCR are well understood by the person skilled in the art, and standard procedures may be used to both detect and to quantify the target molecule. An endogenous target nucleic acid sequence or other exogenous control nucleic acid sequence may be used as a control for concentration measurements, as necessary.

Where it is not necessary to quantify a target nucleic acid molecule, simple hybridisation assays, such as Northern blots, may be carried out to detect and identify particular target sequences within biomarkers or within endogenous genes for genotyping of the individual. Typically, a single-stranded (oligonucleotide) probe is designed to have homology to a target nucleic acid sequence, so that it will hybridise to that target sequence. Beneficially, the probes is labelled in a known manner to allow detection of the bound probe. In the present invention homologous nucleic acid sequences are considered to be those that will hybridise to each other under conditions of low stringency, but are advantageously, those that will hybridise to each other under conditions or high stringency (Sambrook et al., supra). By way of example, for nucleic acid molecules over about 500 bp, stringent hybridisation conditions may include a solution comprising about 1 M Na⁺ at 25° to 30° C. below the melting point, Tm (e.g., 5×SSPE, 0.5% SDS, at 65° C.; see also Ausubel et al., 1995, Current Protocols in Molecular Biology, Greene Publishing).

For the detection and/or quantification of polypeptide molecules alternative techniques may be used, for example, ELISA (luminescent or fluorescent), Biacore systems, and other antibody/antigen recognition techniques.

Antibodies specific for a particular biomarker or therapeutic compound may be selected for use in the methods of the invention according to known techniques. In embodiments of the invention, antibodies or the antigen-binding portions thereof may be used. Antibodies may include: a polyclonal antibody, a monoclonal antibody, a humanised monoclonal antibody derived from a murine monoclonal antibody, and a human monoclonal antibody. The antigen binding portion of an antibody may for example, comprise Fab fragments, F(ab′)2 fragments and Fv fragments.

The antibody or antigen-binding portion thereof may be bound/attached to a label effective to permit detection of the antibody, so that it can be identified and or quantified once bound to its target molecule. Suitably, the biological sample, the solid substrate or testing surface and/or the biomolecule(s) extracted from the substrate is contacted with the antibody or antigen-binding portion thereof having an appropriate label (e.g. a fluorescent label or radioactive marker), under conditions effective to permit binding of the antibody or antigen-binding portion thereof to the polypeptide, polypeptide fragment, or therapeutic compound.

Polyclonal antibodies specific for a predetermined biomarker can be prepared according to standard techniques that are known to the person skilled in the art. Thus, by way of example, a polyclonal antibody that selectively binds to an MRSA protein epitope (for example, a 16 amino acid region), may be raised against an oligopeptide derived from that protein. Alternatively, monoclonal antibodies to polypeptides, or epitope fragments thereof may be prepared, for example, according to the methods described by Galfre & Milstein, 1981, Methods Enzymol., 73(Pt B): 3-46. Most suitably, the epitope of the biomarker (or target molecule) is not destroyed by immobilising the molecule to the solid substrate or, where appropriate, the extraction step.

Monoclonal antibodies may be produced by techniques which are well known in the art, e.g. as described in Kohler & Milstein (1975, Nature, 256: 495). Briefly, immune cells (lymphocytes) are obtained from the spleen of a mammal (e.g. a mouse), which has been previously immunised with the antigen of interest either in vivo or in vitro. The antibody-secreting lymphocytes are then fused with (mouse) myeloma cells or transformed cells, which are able to replicate indefinitely in cell culture, to produce an immortal, immunoglobulin-secreting cell line. The resulting fused cells (i.e. hybridomas), are then cultured, and the resulting colonies are screened for the production of the desired monoclonal antibodies. Colonies producing suitable antibodies are cloned and grown, typically in vitro, to produce a monoclonal antibody.

Mammalian lymphocytes are immunised by in vivo immunisation of the animal (e.g. a mouse) with an antigen, e.g. a biomarker polypeptide or fragment thereof. Immunisations are repeated, as necessary, at intervals of up to a few weeks, to obtain a sufficient titre of antibodies. Following the last antigen injection, the animals are sacrificed and spleen cells removed. Fusion with mammalian myeloma cells, for example, is effected by standard and well-known techniques, e.g. using polyethylene glycol (PEG) or other fusing agents, as described in Milstein & Kohler (1976, Eur. J. Immunol., 6: 511).

Typically, the immortal cell line produced is murine, but may alternatively be derived from cells of other mammalian species, including rats and humans, as known to the person of skill in the art.

Polyclonal antibodies can also be raised using techniques well known in the art. In brief, antigen (as above) is administering subcutaneously to rabbits, for example, which have first been bled to obtain pre-immune serum. The injected material may contain an adjuvant to increase the immune response, for example, a pulverised acrylamide gel containing the antigen may be administered. Rabbits are bled two weeks after the first injection the same antigen is administered three times every six weeks to increase the antibody response. A sample of serum is collected 10 days after each antigen boost, and polyclonal antibodies can be recovered from the serum by affinity chromatography using the corresponding antigen (see e.g. Harlow et al., eds, Antibodies: A Laboratory Manual, 1988).

In addition to whole antibodies, the methods of the invention also encompass antigen-binding portions or fragments of such antibodies. Such antigen-binding portions include Fab fragments, F(ab′)2 fragments, and Fv fragments, which can be made using conventional procedures, such as proteolytic fragmentation (Goding, 1983, Monoclonal Antibodies: Principles and Practice, N.Y. Academic Press, pp 98-118).

A therapeutic compound (and biomarkers, particularly polypeptides) may be detected and/or quantified by any suitable means available to the person skilled in the art, such as those techniques used in TDM. A most suitable means is mass spectrometry according to standard procedures. Such a technique may provide a very accurate measurement of the quantity of the therapeutic molecule concerned.

### Efficacy of a Therapeutic Treatment Regime

Once the relevant biomarkers and therapeutic compound have been detected and/or quantified, the method of the invention may involve the correlation of the amount of a biomarker (that indicates the disease state of the individual), with the amount of the therapeutic compound in the individual's biological sample. The amount of the therapeutic compound may, for example, indicate the bioavailable concentration of the therapeutic compound.

Thus, the method may indicate: (i) the disease state of the individual, i.e. whether the individual is responding well or poorly to the therapeutic treatment regime; (ii) the amount of therapeutic compound in the individual's system, which is available for combating the disease; and (iii) whether the disease state may be linked to the bioavailability of the therapeutic compound in the individual.

Where it is found that the disease is not being adequately managed by the therapeutic treatment regime, for instance, because the level of a biomarker of the disease is higher than expected; this may be because inter alia: (a) the disease is not responding to the therapeutic compound, for example, a pathogen may have developed a level of resistance to the drug; or (b) the individual may be metabolising the therapeutic compound at a faster rate than expected, such that the compound is too rapidly inactivated or removed from the individual's body. The TDM of the therapeutic compound determined in accordance with the methods of the invention may be used to resolve these scenarios.

By way of example, where the TDM of the therapeutic compound is in the correct range (or higher than the correct range), but nevertheless the disease is not being managed as expected, this is a good indication that the disease is not responding to the therapeutic treatment regime and, where there is a pathogenic organism, the pathogen may be developing resistance. In this event, the therapeutic treatment regime may be deemed inefficacious, and a adapted or new regime may be desirable. Typically, such an adapted or new regime may suitably comprise the use of an alternative therapeutic compound.

In contrast, where the TDM of the therapeutic compound is lower than the desirable range and the disease is not being managed as expected, this may be a good indication that the individual is metabolising the therapeutic compound at a higher rate than is normal. In this event, the therapeutic treatment regime may again be deemed inefficacious, and a adapted or new regime may be desirable. However, in this case, it may be advantageous to increase the therapeutic dose of the compound. In such circumstances, the higher rate of metabolism of the therapeutic compound in the individual may be indicative of the individual's ability to tolerate higher doses of the drug; whereas, in the case of a slower drug metabolism level, increasing the drug dose may have toxic consequences.

### Genetic Variability of Individuals and TDM

Another feature of the invention is the possibility of determining a genotype of the individual whom provided the biological sample. This can be useful in, for example, helping to interpret the determined efficacy of the therapeutic treatment regime.

In particular, the efficacy of a therapeutic treatment regime can depend on the amount of bioavailable therapeutic compound (drug) levels in the individual's body, for example, in the blood supply of the patient. In addition to the absolute level (e.g. concentration) of the drug, the duration that the drug remains bioavailable may be important, as previously described. In this regard, for the effective treatment/management of HIV, it can be important to maintain a (relatively constant) steady-state level of the active drug, so that there is not a prolonged duration in which the virus is able to multiply unchecked or infect new cells.

The bioavailability of a therapeutic compound may be influenced by the amount or dose of the compound given to the individual, the size and/or sex of the individual, and the rate of metabolism of the drug by the individual. The rate of metabolism of molecules, such as drugs, can vary considerably in different individuals. Therefore, personalised medicine and treatment regimes can be attractive propositions.

People are known to have different levels of drug metabolism. In recent years, many genes whose products are involved in drug metabolism and, for example, detoxification have been identified. Once particularly important gene/protein has been identified as cytochromes P450 (CYP450). Variants of CYP450 can cause an individual to hypo or hyper-metabolise a particular therapeutic compound: although not necessary for all different therapeutic compounds. If a person hypometabolises a particular compound then, beneficially, the therapeutic dose of that compound in that individual may be lower than for a different person of the same sex, age, ethnicity and weight. If such a person is prescribed a high dose of that compound (or even a normal dosage), this could cause toxicity and in some cases the effects could be fatal. Conversely, if an individual hypermetabolises a therapeutic compound, then a therapeutic dosage for that person may be more than expected or recommended. Otherwise, the drug may have little or no effect in the treatment or management of the respective disease. Thus, by determining a genetic status of an individual with respect to the systems involved in metabolising therapeutic compounds it may be possible for a physician to prescribe drugs in therapeutically effective amounts, while also minimising the risks of drug-related side effects. Furthermore, the cost efficiency of a therapeutic treatment regime may be improved. A particularly suitable therapeutic treatment regime is for the treatment and management of HIV infection and, therefore, it is beneficial to genotype in an individual one or more genes involved in the metabolism of anti-HIV drugs. Advantageously, the genotype of at least one allele of CYP450 is determined in accordance with the invention.

CYP450 (also CYP, P450, and cytochrome P450) is a diverse superfamily of hemoproteins that are found in most organisms including humans, non-human animals, birds, fish, insect, plants, fungi, bacteria and archaea bacteria. In fact, it is thought that there are over 6700 different CYP450 sequences. Significantly, CYP450 is capable of using a vast array of both exogenous and endogenous compounds as substrates in enzymatic reactions. One common reaction catalysed by CYP450 is a monooxygenase reaction, i.e. the insertion of one atom of oxygen into an organic substrate (i.e. RH), while the other oxygen atom is reduced to water: RH+O₂+2^{H}⁺+2e—→ROH+H₂O

CYP450s metabolise thousands of endogenous and exogenous compounds, and most are capable of metabolising multiple substrates. Further, many can catalyse multiple reactions, which accounts for their important role in metabolising the extremely large number of endogenous and exogenous molecules in an individual's body. CYP450 is probably the most important enzyme of oxidative metabolism in humans (and non-human animals). In the liver, CYP450 is responsible for metabolising many drugs and toxic compounds, as well as metabolic products. In other tissues, however, CYP450s play important roles in hormone synthesis and breakdown (including estrogen and testosterone synthesis and metabolism), cholesterol synthesis, and vitamin D metabolism.

The Human Genome Project has identified the sequences of over 63 human CYP450 genes coding for the variant CYP450 enzymes, which means that the person skilled in the art is able to design sequence specific nucleic acid probes for identifying CYP450 alleles and genes such as different isozymes).

There are many drugs and types of drugs that may increase or decrease the activity of the various CYP450 isozymes, and this may an important cause of adverse drug interactions. For example, the inhibition or activation of e.g. liver CYP450 could significantly change the rate of metabolism and clearance of particular therapeutic compounds. Therefore, the way in which an individual's body reacts to one drug may be dramatically changed if that individual is also receiving a second drug, for the same or different therapeutic effect. This can, for example, result in drug overdoses or drug ineffectiveness.

The invention further relates to the genotyping of a particular subset (or subfamily) of CYP450 enzymes, cytochrome P450 3A4 (CYP3A4), which is the main CYP450 expressed in the liver in animals. It is one of the most important enzymes involved in the metabolism of xenobiotics, drugs and steroids (including testosterone) in the body, and it is involved in the oxidation of the largest range of substrates of all the CYPs.

Although CYP3A4 is predominantly found in the liver, it is also present in other organs and tissues of the body where it may play an important role in metabolism. In the intestine, it plays an important role in the metabolism of many drugs, and often it is responsible for allowing prodrugs to be activated and absorbed (e.g. terfenadine).

The CYP3A4 genotype, or at least an allele of CYP3A4 may be identified in accordance with the invention by detecting particular SNPs. Over 28 single SNPs have been identified in the CYP3A4 gene (US2003/0017469).

In some cases, it may be possible to identify the CYP3A4 genotype (or allele) by determining its activity at the enzyme level. For example, CYP3A4 function can be determined by the erythromycin breath test, which estimates in vivo CYP3A4 activity by measuring the radiolabelled carbon dioxide exhaled after an intravenous dose of (14C-N-methyl)-erythromycin (Watkins, 1994, Pharmacogenetics, 4(4): pp 171-84). This can be particularly suitable where the level of expression of CYP3A4 or its enzymatic activity affect the rate of metabolism of a therapeutic compound.

For instance, CYP3A4 expression is induced by a wide variety of ligands, which bind to the Pregnane X Receptor (PXR). The activated PXR complex forms a heterodimer with the Retinoid X Receptor (RXR) that binds to the XREM region of the CYP3A4 gene.

XREM is a regulatory region of the CYP3A4 gene, and binding causes a cooperative interaction with proximal promoter regions of the gene, resulting in increased transcription and expression of CYP3A4. An extremely large number of inducers, inhibitors and substrates of CYP3A4 are known to the person skilled in the art, and include: (i) inducers such as barbiturates (e.g. phenobarbital), glucocorticoids, non-nucleoside reverse transcriptase inhibitors (e.g. efavirenz, modafinil, nevirapine), rifampicin, carbamazepine, dexamethasone, felbamate, hyperforin (constituent of St Johns Wort), griseofulvin, phenytoin, pioglitazone, primidone, topiramate and troglitazone; (ii) inhibitors such as azole antifungals (e.g. ketoconazole, itraconazole), macrolide antibiotics (e.g. erythromycin, telithromycin), bergamottin (in grapefruit juice), cimetidine, ciprofloxacin, non-nucleoside reverse transcriptase inhibitors (e.g. efavirenz, nevirapine), protease inhibitors (e.g. saquinavir, ritonavir, indinavir), selective serotonin reuptake inhibitors (e.g. fluoxetine), amiodarone, aprepitant, ciclosporin, diltiazem, imatinib, echinacea, enoxacin, ergotamine, metronidazole, mifepristone, nefazodone, gestodene, mibefradil, Star fruit, and verapamil; and (iii) substrates such as azole antifungals (e.g. ketoconazole), barbiturates (e.g. phenobarbital), benzodiazepines (e.g. diazepam), calcium channel blockers (e.g. diltiazem, nifedipine), codeine, estradiol, HMG-CoA reductase inhibitors (e.g. atorvastatin), lidocaine, macrolide antibiotics (e.g. erythromycin), mifepristone, ondansetron, omeprazole, paracetamol, quinidine, quinine, taxanes (e.g. paclitaxel), testosterone, amiodarone, buspirone, carbamazepine, ciclosporin, dextromethorphan, digoxin, ergot alkaloids, ethinylestradiol, fentanyl, haloperidol, ivabradine, levonorgestrel, montelukast, PDE5 inhibitors (e.g. sildenafil), selective serotonin reuptake inhibitors (e.g. fluoxetine), theophylline, tricyclic anti-depressants (e.g. amitriptyline), valproate, venlafaxine, warfarin, and various anticancer agents.

### Diagnostic Multiplex Panel

The invention may, in some embodiments, further comprise the high throughput testing of one or more biological samples for the presence of microorganisms in that sample. The high throughput analysis is enabled by the use of a diagnostic multiplexing panel (DMP) that is directed towards genotyping of a plurality of microorganisms that are potentially present in the biological sample, as described in PCT/GB2007/000195 (published as WO 2007/083147).

The DMP provides a combination of primers that each specifically hybridise with a highly conserved sequence in DNA that is isolated from microorganisms that may be present within a biological sample. The DMP allows for simultaneous primer extension reactions to identify if one or more of a plurality of organisms is potentially present in a single sample. The DMPs of the present invention may suitably be directed at particular therapeutic or diagnostic areas, wherein the microorganisms being tested for fall broadly within a disease area or type. In an example of the invention in use described in more detail below, a DMP is assembled directed at diagnosis of the presence of sexually transmitted infection in biological samples taken from human patients. This form of DMP can suitably test for the presence of bacterial pathogens such as Mycoplasma spp.; Chlamydia spp.; Ureaplasma spp; Neisseria spp.; Gardnerella spp.; Trichomonas spp.; Treponema spp; or the yeast Candida albicans; or viral pathogens such as: cytomegalovirus (CMV); hepatitis viruses (e.g. HAV, HBV and HCV); human immunodeficiency viruses (HIV); human papilloma viruses (HPV); herpes simplex viruses (HSV); Molluscum contagiosum virus (MCV); influenza virus; Epstein-Barr virus (EBV) and varicella-zoster virus (VZV).

Other disease areas to which DMPs are suitably directed include: food poisoning; tuberculosis; virally induced cancer; encephalitis; malaria; hepatitis; meningitis; leishmaniasis; African trypanosomiasis; pneumonia; plague; SARS; MRSA; rabies; anthrax; Rift valley fever; tularemia; shigella; botulism; yellow fever; Q fever; ebola; dengue fever; West Nile fever; dysentery; influenza; measles; and typhus.

The invention further enables detection of sequences that confer antibiotic sensitivity in bacterial pathogens by including such sequences in the DMP testing design. This allows speeding up the commencement of treatment of individuals found to be harbouring such pathogens by removing the additional separate step of microbiological identification of antibiotic sensitivity.

Furthermore, the methods of the invention that comprise DMP testing may also be used to provide information about the progression of some diseases by determining the concentration of detected pathogens, which in many cases reflects the progress of the disease. Concentration may include, for example, an assessment of viral load, as in the case of HIV infection. By way of example, quantitative information can be obtained from the primer extension phase of the reaction, for instance, by inclusion of a competitor sequence to a given target sequence, which competitor sequence contains an introduced polymorphism at a specified position in its sequence compared to the target sequence. The competitor sequence can suitably include an alternative nucleotide at the position of a known SNP but which is otherwise identical. If the competitor is supplied during the nucleic acid amplification stage at a known concentration (or copy number) then is can serve as a benchmark for quantifying concentration of the polymorphism-containing target sequence from the microorganism of interest. In one embodiment of the invention it is possible to provide additional competitor sequences at different concentrations (e.g. low, medium and high concentration) all with an introduced sequence variation directed at a specific site in a target sequence to enable more accurate quantification of the microorganism concentration in the original biological sample. Quantification aside, inclusion of competitor sequences also provides an internal control for all the enzymatic steps in diagnostic methods of the invention.

The DMP is suitably provided as a plurality of appropriately plexed primers in solution. However, the DMP can also comprise primers that are immobilised on a solid surface such as in the form of a microarray. The solid surface can suitably be in the form of a silicon substrate or a glass substrate.

Resolution of the DMP reaction products following primer extension can be achieved using a number of technologies, including mass spectrometry (e.g. MALDI-TOF), electrophoresis (e.g. capillary electrophoresis), DNA microarray (e.g. Affymetrix's GeneChip® or printed DNA arrays), via incorporation of fluorescently labelled nucleotides (e.g. Beckman Coulter's SNPstream® or Applied Biosystems' SNPIex®), or other labels (e.g. antigen, biotin, or a radiolabel). The preferred method for resolution of the primer extension products involves determination according to relative molecular weight, both mass spectrometry and capillary electrophoresis are favoured for this.

In one embodiment, each primer comprised within the DMP may vary by overall nucleotide length such that no two primers are of the same relative molecular weight either before or after the primer extension reaction. The products of the reaction are suitably purified in order to optimise mass spectrometric analysis. After purification the products are spotted onto an appropriate element, typically a silicon chip incorporating high-density, photo-resistant array of mass spectrometry analysis sites (e.g. a SpectroCHIP®) and analysed on a matrix assisted laser desorption/ionisation-time-of-flight (MALDI-TOF) mass spectrometer (e.g. Sequenom's MassArray® Mass

Spectrometer as described in U.S. Pat. Nos. 6,500,621, 6,300,076, 6,258,538, 5,869,242, 6,238,871, 6,440,705, and 6,994,969). The results of mass spectrometric analysis may be processed using an appropriate software package, so as to provide information on the presence or absence of primer extension products that are correlated to the presence or absence of particular specified microorganisms in the biological sample.

### EXPERIMENTAL

### Materials and Methods

The practice of the present invention will employ, unless otherwise indicated, conventional techniques of chemistry, molecular biology, microbiology, recombinant DNA and immunology, which are within the capabilities of a person of ordinary skill in the art. In addition, such techniques are explained in the literature, for example: J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, & A. Kahn, 1996, DNA Isolation and Sequencing: Essential Techniques, John Wiley & Sons; J. M. Polak & James O'D. McGee, 1990, In Situ Hybridization: Principles and Practice; Oxford University Press; M. J. Gait (Editor), 1984, Oligonucleotide Synthesis: A Practical Approach, Irl Press; and D. M. J. Lilley & J. E. Dahlberg, 1992, Methods of Enzymology: DNA Structure Part A: Synthesis and Physical Analysis of DNA Methods in Enzymology, Academic Press.

### EXAMPLE

The methods of the invention are used to monitor a therapeutic treatment regime for a patient infected with HIV. A blood sample or approximately 50 μl is obtained from the individual at a first non-clinical location, as previously described, and placed onto a solid substrate capable of immobilising HIV RNA genome and the anti-HIV therapeutic compound or compounds in use.

The anti-HIV therapeutic compound(s) may be a protease inhibitor of HIV protease, and/or a reverse transcriptase (RT) inhibitor of HIV RT. The solid substrate also inactivates the HIV within the blood sample.

A device in accordance with the invention containing a solid substrate sealed inside a chamber is posted using a public postal service to a testing facility, such as a laboratory.

At the laboratory the device is broken open by an authorised person to obtain the solid support with its immobilised molecules thereon and therein. The HIV nucleic acid (RNA) and the anti-HIV therapeutic compound(s) are extracted by boiling the solid support in an aqueous buffer.

The concentration of HIV genome in the blood sample is determined using techniques known to the person skilled in the art. Likewise, the concentration of any anti-HIV therapeutic compound(s) is determined, for example, using mass spectrophotometry.

Optionally, a genotype of the individual is also determined, for example, a CYP450 allele (e.g. a CYP3A4 allele).

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1.-44. (canceled)

45. A method for monitoring a therapeutic treatment regime in an individual having a disease, comprising detecting and/or measuring the concentration of a biomarker characteristic of the disease and a therapeutic compound in a biological sample obtained from the individual, the method comprising the steps of:
(a) providing at a first location at least one solid substrate, the at least one solid substrate capable of immobilising at least the biomarker and the therapeutic compound in the biological sample, and contacting the biological sample with the at least one solid substrate to immobilise the biomarker and the therapeutic compound on and/or within the solid substrate at the first location;
(b) transferring the solid substrate with the immobilised biomarker and therapeutic compound to a second location;
(c) performing an extraction step on the solid substrate to extract at least the biomarker and the therapeutic compound;
(d) performing a first detection assay to detect and/or quantify the biomarker, and performing a second detection assay to quantify the therapeutic compound; and
(e) correlating the detection and/or quantity of the biomarker with the disease state of the individual, and comparing the quantity of the therapeutic compound with a target level of the therapeutic compound for treatment of the disease, to assess the efficacy of the treatment regime.

46. The method of claim 45, wherein in step (b) the transfer is carried out by a public postal service.

47. The method of claim 45, wherein the biomarker is characteristic of a pathogenic microorganism against which the therapeutic compound is directed.

48. The method of claim 45, wherein the disease is a sexually transmitted infection and the biomarker is associated with one or more microorganisms selected from the group consisting of: Mycoplasma genitalum, Mycoplasma hominis, Chlamydia trachomatis, Ureaplasma urealyticum, Neisseria gonorrhoea, Gardnerella vaginalis, Trichomonas vaginalis, Treponema pallidum, CMV, HAV, HBV, HCV, HEV, GBV-C, HIV-1, HIV-2, HPV, HSV-1, HSV-2, MCV, VZV and EBV.

49. The method of claim 48, wherein the biomarker is associated with HIV and the therapeutic compound is an anti-HIV drug.

50. The method of claim 45, wherein the biomarker is a nucleic acid molecule and in step (d) the first detection assay comprises a nucleic acid amplification step; and wherein the nucleic acid application is directed towards a target sequence of the nucleic acid molecule.

51. The method of claim 50, wherein the nucleic acid amplification step comprises a plurality of pairs of nucleic acid amplification primers that are adapted for amplification of a plurality of target sequences in nucleic acid molecules from one or more microorganism.

52. The method of claim 45, wherein the solid substrate comprises an absorbent fibrous material and one or more reagents that immobilise and inactivate any microorganisms that may be present in the biological sample.

53. The method of claim 45, wherein the biomarker is a highly conserved polymorphic allele selected from the group consisting of: a single nucleotide polymorphism (SNP), an insertion, a deletion, an inversion, and a substitution.

54. The method of claim 45, wherein the biological sample comprises at least one of the group consisting of: urine, saliva, blood, sputum, semen, faeces, a nasal swab, tears, a vaginal swab, a rectal swab, a cervical smear, a tissue biopsy, and a urethral swab.

55. The method of claim 45, wherein the solid substrate is further capable of immobilising endogenous nucleic acid molecules present in the biological sample, and the method further comprises the step of determining a genotype of the individual based on an endogenous nucleic acid molecule immobilised on the solid substrate.

56. The method of claim 55, wherein the step of determining a genotype of the individual comprises: performing a nucleic acid amplification step on the endogenous nucleic acid, wherein the nucleic acid amplification is directed towards at least one target sequence of one or more endogenous gene.

57. The method of claim 56, wherein the one or more endogenous genes encodes a protein involved in metabolising the therapeutic compound.

58. The method of claim 57, wherein the endogenous gene is a cytochrome P450 gene.

59. A method for determining the drug sensitivity of an individual undergoing a therapeutic treatment regime, comprising measuring the concentration of a therapeutic compound in a biological sample obtained from the individual, and determining a genotype of the individual, the method comprising the steps of:
(a) providing at a first location at least one solid substrate, the at least one solid substrate capable of immobilising at least the therapeutic compound and endogenous nucleic acid molecules in the biological sample, and contacting the biological sample with the at least one solid substrate to immobilise the therapeutic compound and the endogenous nucleic acid on and/or within the solid substrate at the first location;
(b) transferring the solid substrate with the immobilised therapeutic compound and endogenous nucleic acid molecules to a second location;
(c) performing a extraction step on the solid substrate to extract at least the therapeutic compound and the endogenous nucleic acid molecules immobilised on and/or within the solid substrate; and
(d) performing a first detection assay to quantify the therapeutic compound and performing a second detection assay on the endogenous nucleic acid molecules to identify a genotype of one or more endogenous gene of the individual.

60. The method of claim 59, wherein at least one of the one or more endogenous genes is an endogenous biomarker of a disease.

61. A device for use in monitoring a therapeutic treatment regime in an individual having a disease comprising detecting and/or measuring the concentration of a biomarker characteristic of the disease and a therapeutic compound in a biological sample obtained from the individual, the device comprising:
a testing surface located within a sealable chamber, the testing surface comprising a solid substrate that is capable of immobilising the biological sample, including at least the biomarker and the therapeutic compound, either within it or upon its surface; and
wherein, in use, once a biological sample has been deposited upon the testing surface, the chamber is sealable around the testing surface such that the biological sample is enclosed within the chamber.

62. The device of claim 61, wherein the solid substrate comprises an absorbent matrix that comprises one or more reagents that immobilise and inactivate any microorganisms present in the biological sample.

63. The device of claim 61, wherein the testing surface comprises a solid substrate comprising two or more different materials and/or two or more different reagents that immobilise and inactivate any microorganisms present in the biological sample.

64. The device of claim 61, wherein the solid substrate is further capable of immobilising endogenous nucleic acid molecules in the biological sample.
